SEQUENCE LISTING (17 sequences)

SEQ ID NO: 1 - protein, 157 aa, Artificial sequence
  E6 protein of HPV16
HQKRTAMFQDPQERPRKLPQLCTELQTTIHDIILECVYCKQQLLRREVYDFAFRDLCIVYRDGNPYAVGDKCLKFYSKISEYRHYCYSLYGTTLEQQYNKPLCDLLIRCINCQKPLCPEEKQRHLDKKQRFHNTRGRWTGRCMSCCRSSRTRRETQL

SEQ ID NO: 2 - protein, 100 aa, Artificial sequence
  E7 protein of HPV16
HGDTPTLHEYMLDLQPETTDLYCGSGYEQLNDSSEEEDEIDGPAGQAEPDRAHYNIVTFCCKCDSTLRLCVQSTHVDIRTLEDLLMGTLGIVCPICSQKP

SEQ ID NO: 3 - protein, 98 aa, Artificial sequence
  CCL13
MKVSAVLLCLLLMTAAFNPQGLAQPDALNVPSTCCFTFSSKKISLQRLKSYVITTSRCPQKAVIFRTKLGKEICADPKEKWVQNYMKHLGRKAHTLKT

SEQ ID NO: 4 - protein, 31 aa, Artificial sequence
  T2 fragment
PGSGYIPEAPRDGQAYVRKDGEWVLLSTFLG

SEQ ID NO: 5 - protein, 18 aa, Artificial sequence
  IgE signal peptide
MDWTWILFLVAAATRVHS

SEQ ID NO: 6 - DNA, 4595 bp, Artificial sequence
  pVR-CCL13-E6E7-T2
gactcttcgcgatgtacgggccagatatacgcgttccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctactctggctaactagagaacccactgcttactggcttatcgaaattaatacgactcactatagggagacccaagctggctagcgtttaaacttaagcttggtaccgagctcggatccgccaccatggactggacctggatcctgttcctggtggccgccgccacaagggtgcacagcaaagtctctgcagtgcttctgtgcctgctgctcatgacagcagctttcaacccccagggacttgctcagccagatgcactcaacgtcccatctacttgctgcttcacatttagcagtaagaagatctccttgcagaggctgaagagctatgtgatcaccaccagcaggtgtccccagaaggctgtcatcttcagaaccaaactgggcaaggagatctgtgctgacccaaaggagaagtgggtccagaattatatgaaacacctgggccggaaagctcacaccctgaagacttgacaccagaagaggacagcaatgttccaggacccacaggagaggccaaggaagctgccacagctgtgcacagagctgcagacaacaatccacgatattatcctggagtgcgtgtattgcaagcagcagctgctgaggagggaggtgtatgacttcgctttcagagacctgtgtatcgtgtaccgggacgggaacccttacgcagtgggcgacaagtgcctgaagttctatagcaagatcagcgagtacaggcactactgttacagcctgtatggaaccaccctggagcagcagtataacaaacctctgtgcgacctgctgatcaggtgcatcaactgccagaagcccctgtgccccgaggagaagcagagacacctggacaagaagcagaggttccacaataccaggggccgctggaccggccgctgcatgtcttgctgcaggtccagccggaccaggagagagacccagctggccggagcacacggagatacacccaccctgcacgagtacatgctggacctgcagcctgagaccaccgacctgtactgcgggagtggatacgaacagctgaacgacagcagcgaggaggaggacgagatcgacggccccgccggacaggctgagcctgatagggcccactacaacattgtgacattttgctgcaagtgcgacagcaccctgaggctgtgcgtgcagagcacccacgtggatatcaggaccctggaggatctgctgatgggaaccctgggcatcgtgtgccccatctgctcccagaagcctcctggatctggctacattcctgaagcccctcgcgacggccaggcctacgtgagaaaagacggcgaatgggtgctgctgagcacctttctgggcgactacaaggacgacgacgacaagtgatgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 7 - DNA, 4502 bp, Artificial sequence
  pVR-CCL13-E6E7
gactcttcgcgatgtacgggccagatatacgcgttccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctactctggctaactagagaacccactgcttactggcttatcgaaattaatacgactcactatagggagacccaagctggctagcgtttaaacttaagcttggtaccgagctcggatccgccaccatggactggacctggatcctgttcctggtggccgccgccacaagggtgcacagcaaagtctctgcagtgcttctgtgcctgctgctcatgacagcagctttcaacccccagggacttgctcagccagatgcactcaacgtcccatctacttgctgcttcacatttagcagtaagaagatctccttgcagaggctgaagagctatgtgatcaccaccagcaggtgtccccagaaggctgtcatcttcagaaccaaactgggcaaggagatctgtgctgacccaaaggagaagtgggtccagaattatatgaaacacctgggccggaaagctcacaccctgaagacttgacaccagaagaggacagcaatgttccaggacccacaggagaggccaaggaagctgccacagctgtgcacagagctgcagacaacaatccacgatattatcctggagtgcgtgtattgcaagcagcagctgctgaggagggaggtgtatgacttcgctttcagagacctgtgtatcgtgtaccgggacgggaacccttacgcagtgggcgacaagtgcctgaagttctatagcaagatcagcgagtacaggcactactgttacagcctgtatggaaccaccctggagcagcagtataacaaacctctgtgcgacctgctgatcaggtgcatcaactgccagaagcccctgtgccccgaggagaagcagagacacctggacaagaagcagaggttccacaataccaggggccgctggaccggccgctgcatgtcttgctgcaggtccagccggaccaggagagagacccagctggccggagcacacggagatacacccaccctgcacgagtacatgctggacctgcagcctgagaccaccgacctgtactgcgggagtggatacgaacagctgaacgacagcagcgaggaggaggacgagatcgacggccccgccggacaggctgagcctgatagggcccactacaacattgtgacattttgctgcaagtgcgacagcaccctgaggctgtgcgtgcagagcacccacgtggatatcaggaccctggaggatctgctgatgggaaccctgggcatcgtgtgccccatctgctcccagaagcctgactacaaggacgacgacgacaagtgatgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 8 - DNA, 4208 bp, Artificial sequence
  pVR-E6E7
gactcttcgcgatgtacgggccagatatacgcgttccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctactctggctaactagagaacccactgcttactggcttatcgaaattaatacgactcactatagggagacccaagctggctagcgtttaaacttaagcttggtaccgagctcggatccgccaccatggactggacctggatcctgttcctggtggccgccgccacaagggtgcacagccaccagaagaggacagcaatgttccaggacccacaggagaggccaaggaagctgccacagctgtgcacagagctgcagacaacaatccacgatattatcctggagtgcgtgtattgcaagcagcagctgctgaggagggaggtgtatgacttcgctttcagagacctgtgtatcgtgtaccgggacgggaacccttacgcagtgggcgacaagtgcctgaagttctatagcaagatcagcgagtacaggcactactgttacagcctgtatggaaccaccctggagcagcagtataacaaacctctgtgcgacctgctgatcaggtgcatcaactgccagaagcccctgtgccccgaggagaagcagagacacctggacaagaagcagaggttccacaataccaggggccgctggaccggccgctgcatgtcttgctgcaggtccagccggaccaggagagagacccagctggccggagcacacggagatacacccaccctgcacgagtacatgctggacctgcagcctgagaccaccgacctgtactgcgggagtggatacgaacagctgaacgacagcagcgaggaggaggacgagatcgacggccccgccggacaggctgagcctgatagggcccactacaacattgtgacattttgctgcaagtgcgacagcaccctgaggctgtgcgtgcagagcacccacgtggatatcaggaccctggaggatctgctgatgggaaccctgggcatcgtgtgccccatctgctcccagaagcctgactacaaggacgacgacgacaagtgatgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 9 - DNA, 1194 bp, Artificial sequence
  fusion protein in plasmid pVR-CCL13-E6E7-T2
aaagtctctgcagtgcttctgtgcctgctgctcatgacagcagctttcaacccccagggacttgctcagccagatgcactcaacgtcccatctacttgctgcttcacatttagcagtaagaagatctccttgcagaggctgaagagctatgtgatcaccaccagcaggtgtccccagaaggctgtcatcttcagaaccaaactgggcaaggagatctgtgctgacccaaaggagaagtgggtccagaattatatgaaacacctgggccggaaagctcacaccctgaagacttgacaccagaagaggacagcaatgttccaggacccacaggagaggccaaggaagctgccacagctgtgcacagagctgcagacaacaatccacgatattatcctggagtgcgtgtattgcaagcagcagctgctgaggagggaggtgtatgacttcgctttcagagacctgtgtatcgtgtaccgggacgggaacccttacgcagtgggcgacaagtgcctgaagttctatagcaagatcagcgagtacaggcactactgttacagcctgtatggaaccaccctggagcagcagtataacaaacctctgtgcgacctgctgatcaggtgcatcaactgccagaagcccctgtgccccgaggagaagcagagacacctggacaagaagcagaggttccacaataccaggggccgctggaccggccgctgcatgtcttgctgcaggtccagccggaccaggagagagacccagctggccggagcacacggagatacacccaccctgcacgagtacatgctggacctgcagcctgagaccaccgacctgtactgcgggagtggatacgaacagctgaacgacagcagcgaggaggaggacgagatcgacggccccgccggacaggctgagcctgatagggcccactacaacattgtgacattttgctgcaagtgcgacagcaccctgaggctgtgcgtgcagagcacccacgtggatatcaggaccctggaggatctgctgatgggaaccctgggcatcgtgtgccccatctgctcccagaagcctcctggatctggctacattcctgaagcccctcgcgacggccaggcctacgtgagaaaagacggcgaatgggtgctgctgagcacctttctgggcgactacaaggacgacgacgacaagtga

SEQ ID NO: 10 - DNA, 1101 bp, Artificial sequence
  fusion protein in plasmid pVR-CCL13-E6E7
aaagtctctgcagtgcttctgtgcctgctgctcatgacagcagctttcaacccccagggacttgctcagccagatgcactcaacgtcccatctacttgctgcttcacatttagcagtaagaagatctccttgcagaggctgaagagctatgtgatcaccaccagcaggtgtccccagaaggctgtcatcttcagaaccaaactgggcaaggagatctgtgctgacccaaaggagaagtgggtccagaattatatgaaacacctgggccggaaagctcacaccctgaagacttgacaccagaagaggacagcaatgttccaggacccacaggagaggccaaggaagctgccacagctgtgcacagagctgcagacaacaatccacgatattatcctggagtgcgtgtattgcaagcagcagctgctgaggagggaggtgtatgacttcgctttcagagacctgtgtatcgtgtaccgggacgggaacccttacgcagtgggcgacaagtgcctgaagttctatagcaagatcagcgagtacaggcactactgttacagcctgtatggaaccaccctggagcagcagtataacaaacctctgtgcgacctgctgatcaggtgcatcaactgccagaagcccctgtgccccgaggagaagcagagacacctggacaagaagcagaggttccacaataccaggggccgctggaccggccgctgcatgtcttgctgcaggtccagccggaccaggagagagacccagctggccggagcacacggagatacacccaccctgcacgagtacatgctggacctgcagcctgagaccaccgacctgtactgcgggagtggatacgaacagctgaacgacagcagcgaggaggaggacgagatcgacggccccgccggacaggctgagcctgatagggcccactacaacattgtgacattttgctgcaagtgcgacagcaccctgaggctgtgcgtgcagagcacccacgtggatatcaggaccctggaggatctgctgatgggaaccctgggcatcgtgtgccccatctgctcccagaagcctgactacaaggacgacgacgacaagtga

SEQ ID NO: 11 - DNA, 780 bp, Artificial sequence
  fusion protein in plasmid pVR-E6E7
caccagaagaggacagcaatgttccaggacccacaggagaggccaaggaagctgccacagctgtgcacagagctgcagacaacaatccacgatattatcctggagtgcgtgtattgcaagcagcagctgctgaggagggaggtgtatgacttcgctttcagagacctgtgtatcgtgtaccgggacgggaacccttacgcagtgggcgacaagtgcctgaagttctatagcaagatcagcgagtacaggcactactgttacagcctgtatggaaccaccctggagcagcagtataacaaacctctgtgcgacctgctgatcaggtgcatcaactgccagaagcccctgtgccccgaggagaagcagagacacctggacaagaagcagaggttccacaataccaggggccgctggaccggccgctgcatgtcttgctgcaggtccagccggaccaggagagagacccagctggccggagcacacggagatacacccaccctgcacgagtacatgctggacctgcagcctgagaccaccgacctgtactgcgggagtggatacgaacagctgaacgacagcagcgaggaggaggacgagatcgacggccccgccggacaggctgagcctgatagggcccactacaacattgtgacattttgctgcaagtgcgacagcaccctgaggctgtgcgtgcagagcacccacgtggatatcaggaccctggaggatctgctgatgggaaccctgggcatcgtgtgccccatctgctcccagaagcct

SEQ ID NO: 12 - RNA, 1194 nt, Artificial sequence
  fusion protein in plasmid pVR-CCL13-E6E7-T2
aaagucucugcagugcuucugugccugcugcucaugacagcagcuuucaacccccagggacuugcucagccagaugcacucaacgucccaucuacuugcugcuucacauuuagcaguaagaagaucuccuugcagaggcugaagagcuaugugaucaccaccagcagguguccccagaaggcugucaucuucagaaccaaacugggcaaggagaucugugcugacccaaaggagaaguggguccagaauuauaugaaacaccugggccggaaagcucacacccugaagacuugacaccagaagaggacagcaauguuccaggacccacaggagaggccaaggaagcugccacagcugugcacagagcugcagacaacaauccacgauauuauccuggagugcguguauugcaagcagcagcugcugaggagggagguguaugacuucgcuuucagagaccuguguaucguguaccgggacgggaacccuuacgcagugggcgacaagugccugaaguucuauagcaagaucagcgaguacaggcacuacuguuacagccuguauggaaccacccuggagcagcaguauaacaaaccucugugcgaccugcugaucaggugcaucaacugccagaagccccugugccccgaggagaagcagagacaccuggacaagaagcagagguuccacaauaccaggggccgcuggaccggccgcugcaugucuugcugcagguccagccggaccaggagagagacccagcuggccggagcacacggagauacacccacccugcacgaguacaugcuggaccugcagccugagaccaccgaccuguacugcgggaguggauacgaacagcugaacgacagcagcgaggaggaggacgagaucgacggccccgccggacaggcugagccugauagggcccacuacaacauugugacauuuugcugcaagugcgacagcacccugaggcugugcgugcagagcacccacguggauaucaggacccuggaggaucugcugaugggaacccugggcaucgugugccccaucugcucccagaagccuccuggaucuggcuacauuccugaagccccucgcgacggccaggccuacgugagaaaagacggcgaaugggugcugcugagcaccuuucugggcgacuacaaggacgacgacgacaaguga

SEQ ID NO: 13 - RNA, 1101 nt, Artificial sequence
  fusion protein in plasmid pVR-CCL13-E6E7
aaagucucugcagugcuucugugccugcugcucaugacagcagcuuucaacccccagggacuugcucagccagaugcacucaacgucccaucuacuugcugcuucacauuuagcaguaagaagaucuccuugcagaggcugaagagcuaugugaucaccaccagcagguguccccagaaggcugucaucuucagaaccaaacugggcaaggagaucugugcugacccaaaggagaaguggguccagaauuauaugaaacaccugggccggaaagcucacacccugaagacuugacaccagaagaggacagcaauguuccaggacccacaggagaggccaaggaagcugccacagcugugcacagagcugcagacaacaauccacgauauuauccuggagugcguguauugcaagcagcagcugcugaggagggagguguaugacuucgcuuucagagaccuguguaucguguaccgggacgggaacccuuacgcagugggcgacaagugccugaaguucuauagcaagaucagcgaguacaggcacuacuguuacagccuguauggaaccacccuggagcagcaguauaacaaaccucugugcgaccugcugaucaggugcaucaacugccagaagccccugugccccgaggagaagcagagacaccuggacaagaagcagagguuccacaauaccaggggccgcuggaccggccgcugcaugucuugcugcagguccagccggaccaggagagagacccagcuggccggagcacacggagauacacccacccugcacgaguacaugcuggaccugcagccugagaccaccgaccuguacugcgggaguggauacgaacagcugaacgacagcagcgaggaggaggacgagaucgacggccccgccggacaggcugagccugauagggcccacuacaacauugugacauuuugcugcaagugcgacagcacccugaggcugugcgugcagagcacccacguggauaucaggacccuggaggaucugcugaugggaacccugggcaucgugugccccaucugcucccagaagccugacuacaaggacgacgacgacaaguga

SEQ ID NO: 14 - RNA, 780 nt, Artificial sequence
  fusion protein in plasmid pVR-E6E7
caccagaagaggacagcaauguuccaggacccacaggagaggccaaggaagcugccacagcugugcacagagcugcagacaacaauccacgauauuauccuggagugcguguauugcaagcagcagcugcugaggagggagguguaugacuucgcuuucagagaccuguguaucguguaccgggacgggaacccuuacgcagugggcgacaagugccugaaguucuauagcaagaucagcgaguacaggcacuacuguuacagccuguauggaaccacccuggagcagcaguauaacaaaccucugugcgaccugcugaucaggugcaucaacugccagaagccccugugccccgaggagaagcagagacaccuggacaagaagcagagguuccacaauaccaggggccgcuggaccggccgcugcaugucuugcugcagguccagccggaccaggagagagacccagcuggccggagcacacggagauacacccacccugcacgaguacaugcuggaccugcagccugagaccaccgaccuguacugcgggaguggauacgaacagcugaacgacagcagcgaggaggaggacgagaucgacggccccgccggacaggcugagccugauagggcccacuacaacauugugacauuuugcugcaagugcgacagcacccugaggcugugcgugcagagcacccacguggauaucaggacccuggaggaucugcugaugggaacccugggcaucgugugccccaucugcucccagaagccu

SEQ ID NO: 15 - protein, 359 aa, Artificial sequence
  fusion protein in plasmid pVR-CCL13-E6E7-T2
SPYSSDTTPCCFAYIARPLPRAHIKEYFYTSGKCSNPAVVFVTRKNRQVCANPEKKWVREYINSLEMFQDPQERPRKLPQLCTELQTTIHDIILECVYCKQQLLRREVYDFAFRDLCIVYRDGNPYAVGDKCLKFYSKISEYRHYCYSLYGTTLEQQYNKPLCDLLIRCINCQKPLCPEEKQRHLDKKNRFHNTRGRWTGRCMSCCRSSRTRRETQLAGAHGDTPTLHEYMLDLQPETTDLYCGSGYEQLNDSSEEEDEIDGPAGQAEPDRAHYNIVTFCCKCDSTLRLCVQSTHVDIRTLEDLLMGTLGIVCPICSQKPPGSGYIPEAPRDGQAYVRKDGEWVLLSTFLGDYKDDDDK

SEQ ID NO: 16 - protein, 320 aa, Artificial sequence
  fusion protein in plasmid pVR-CCL13-E6E7
SPYSSDTTPCCFAYIARPLPRAHIKEYFYTSGKCSNPAVVFVTRKNRQVCANPEKKWVREYINSLEMFQDPQERPRKLPQLCTELQTTIHDIILECVYCKQQLLRREVYDFAFRDLCIVYRDGNPYAVGDKCLKFYSKISEYRHYCYSLYGTTLEQQYNKPLCDLLIRCINCQKPLCPEEKQRHLDKKNRFHNTRGRWTGRCMSCCRSSRTRRETQLAGAHGDTPTLHEYMLDLQPETTDLYCGSGYEQLNDSSEEEDEIDGPAGQAEPDRAHYNIVTFCCKCDSTLRLCVQSTHVDIRTLEDLLMGTLGIVCPICSQKP

SEQ ID NO: 17 - protein, 260 aa, Artificial sequence
  fusion protein in plasmid pVR-E6E7
HQKRTAMFQDPQERPRKLPQLCTELQTTIHDIILECVYCKQQLLRREVYDFAFRDLCIVYRDGNPYAVGDKCLKFYSKISEYRHYCYSLYGTTLEQQYNKPLCDLLIRCINCQKPLCPEEKQRHLDKKQRFHNTRGRWTGRCMSCCRSSRTRRETQLAGAHGDTPTLHEYMLDLQPETTDLYCGSGYEQLNDSSEEEDEIDGPAGQAEPDRAHYNIVTFCCKCDSTLRLCVQSTHVDIRTLEDLLMGTLGIVCPICSQKP

(19) United States
(12) Patent Application Publication
(10) Pub. No.: US 2025/0049916 A1
(43) Pub. Date: Feb. 13, 2025

(54) USE OF CCL13

(71) Applicants: NEWISH TECHNOLOGY (BEIJING) CO., LTD., Beijing (CN); INSTITUTE OF MEDICINAL BIOTECHNOLOGY, CHINESE ACADEMY OF MEDICAL SCIENCES, Beijing (CN)

(72) Inventors: Xiaofang WANG, Beijing (CN); Jiandong JIANG, Beijing (CN); Yanling YAO, Beijing (CN); Zhongjie SUN, Beijing (CN); Lulu WANG, Beijing (CN); Xudong WANG, Beijing (CN); Defang LIU, Beijing (CN); Hailong QI, Beijing (CN)

(73) Assignees: NEWISH TECHNOLOGY (BEIJING) CO., LTD., Beijing (CN); INSTITUTE OF MEDICINAL BIOTECHNOLOGY, CHINESE ACADEMY OF MEDICAL SCIENCES, Beijing (CN)

(21) Appl. No.: 18/719,415

(22) PCT Filed: Jun. 6, 2022
(86) PCT No.: PCT/CN2022/097091

§ 371 Date: Jun. 13, 2024

(30) Foreign Application Priority Data
Jan. 24, 2022 (CN) 202210076816.7

(51) Int. Cl. A61K 39/39; A61K 39/12; C07K 14/52; C12N 7/00; C12N 15/62

(52) U.S. Cl. CPC A61K 39/39; A61K 39/12; C07K 14/523; C12N 7/00; C12N 15/62; C12N 2710/20022; C12N 2710/20034

(57) ABSTRACT

The chemotactic binding ability of CCL13 and an immune cell surface receptor such as DC cells is used to transport different antigenic proteins to the surface of the DC cells, the efficiency of phagocytosis, processing, and presentation of various antigenic proteins by the DC cells is improved, and the effect of preventing and treating related diseases of the antigenic proteins is enhanced. A T2 sequence is further added in an antigen, such that the immune effect can be enhanced.

This application claims the priority of Chinese Patent Application No. 202210076816.7, filed with the China National Intellectual Property Administration on Jan. 24, 2022, and titled with “USE OF CCL5”, which is hereby incorporated by reference in its entirety.

### FIELD

The present disclosure relates to the field of biomedicine, and in particular to use of CCL13.

### BACKGROUND

Monocyte chemoattractant protein 4 (MCP-4/CCL13) is a member of a distinct, structurally related CC chemokine subfamily. CCL13 is a major chemoattractant for eosinophils, basophils, monocytes, and T lymphocytes, and can induce key immune regulatory responses through actions on epithelial cells, muscle cells, and endothelial cells. CCL13 is implicated in many chronic inflammatory diseases where it functions as a key molecule involved in the selective recruitment of cell lineages to inflamed tissues and the subsequent activation. In addition, CCL13 plays an important role in the migration of dendritic cells (DCs) to inflamed epithelial layers, and when injected in vivo with an antigen, CCL13 recruits blood monocytes or blood DC precursors which rapidly differentiate into typical DCs, and improves anti-tumor immune response.

Tumor vaccines induce the effector T cells in patients to exert functions by potentiating the existing anti-tumor response or activating naive T cells. Antigen-specific CD8+ cytotoxic T lymphocytes (CTL) play an important role in the anti-tumor process. DCs are the only professional antigen-presenting cells that can activate naive CD8+ T cells. They uptake, process and cross-present extracellular tumor antigens through MHC-I, and are crucial for the production of effective CTLs. Therefore, delivery of tumor antigens to DCs by conjugating DC cell surface molecules is an effective tumor therapy strategy for inducing CD8+ T cell immune responses.

The molecules that have been identified by research to enhance the presentation of DCs include XCL-1, but it is still necessary to develop more DC cell surface molecules capable of enhancing presentation.

### SUMMARY

In view of this, the technical problem to be solved by the present disclosure is to provide an immune enhancement delivery system formed by a T2 fragment and/or CCL13 targeting delivering antigen.

The present disclosure provides:

Use of at least one of I)-VI) in improving antigen presentation effect;
I) a T2 fragment with an amino acid sequence set forth in SEQ ID NO: 4;
II) chemokine CCL13;
III) a fragment being more than 80% homologous with and functionally identical or similar to I) or II);
IV) a nucleic acid molecule encoding I) or II);
V) a nucleic acid molecule that is derived from the nucleotide sequence of the nucleic acid molecule in IV) by deletion, addition or substitution of one or more nucleotides and encode a protein functionally identical or similar to the nucleic acid molecule in IV);
VI) a nucleic acid molecule fully or partially complementary to V).

In the present disclosure, the T2 fragment consists of 31 amino acids, and has a sequence set forth in SEQ ID NO: 4. Studies have shown that the artificially modified T2 fragment has the effect of enhancing immunity in the human body.

In the present disclosure, the CCL13 is a chemokine CCL13 derived from humans or other animals, and is a full-length sequence or a fragment with CCL13 activity. In the present disclosure, it is found that CCL13 can deliver antigens to DCs, thereby improving the presentation effect and enhancing immune response.

The present disclosure also provides a fusion protein comprising CCL13 and an antigen or comprising CCL13, an antigen and a T2 fragment.

In an embodiment of the present disclosure, the fusion protein, from N-terminal to C-terminal, sequentially comprises an IgE signal peptide, CCL13, a linker, the antigen and the T2 fragment.

In the present disclosure, the antigen is derived from a virus, pathogenic microbe and/or tumor.

In the present disclosure, the CCL13 is a humanized CCL13 sequence, and the antigen is E6 protein of HPV16 and/or E7 protein of HPV16.

In some embodiments, the fusion protein, from N-terminal to C-terminal, sequentially comprises an IgE signal peptide, CCL13, a linker, E6 protein of HPV16, E7 protein of HPV16, and the T2 fragment.

In some specific embodiments, the IgE signal peptide has an amino acid sequence set forth in SEQ ID NO: 5;
the CCL13 has an amino acid sequence set forth in SEQ ID NO: 3;
the linker is (GsS) n, wherein n is 1-10; in the embodiments of the present disclosure, the linker has a sequence of GGGGGSGGGGG.

The E6 protein of HPV16 has an amino acid sequence set forth in SEQ ID NO: 1.

The E7 protein of HPV16 has an amino acid sequence set forth in SEQ ID NO: 2.

The T2 fragment has an amino acid sequence set forth in SEQ ID NO: 4.

In some specific embodiments, the fusion protein further comprises a FLAG tag sequence at the C-terminal with an amino acid sequence of DYKDDDDK, which is only a tag for identifying protein expression and does not affect the immune effect of the sequence.

The present disclosure further provides a nucleic acid encoding the fusion protein.

The nucleic acid encoding the fusion protein of the present disclosure has a nucleotide sequence set forth in SEQ ID NO: 9.

The present disclosure provides a nucleic acid fragment, which comprises the nucleic acid encoding the fusion protein of the present disclosure, 5′-UTR, 3′-UTR and 3′-end polyA, wherein the 5′-UTR is β-globin-2, the 3′-UTR is 2β-globin, and the 3′-end polyA has a length of 120 bp. The nucleic acid fragment has a structure of 5′UTR-CCL13-E6E7-3′UTR-A (120).

The present disclosure also provides a transcription unit encoding the fusion protein.

The transcription unit comprises a promoter and the nucleic acid encoding the fusion protein.

In some embodiments, the transcription unit further comprises a terminator.

In some specific embodiments, the promoter is a CMV or CMV/R promoter.

The present disclosure further provides an expression vector, comprising a vector backbone and the nucleic acid encoding the fusion protein.

In the present disclosure, the vector backbone is selected from the group consisting of pVAX1 series vectors and pVR series vectors.

The present disclosure further provides a recombinant host transformed or transfected with the expression vector.

In the present disclosure, the host cells of the recombinant host are bacteria or

mammalian cells.

The present disclosure provides a method for producing the fusion protein, comprising culturing the recombinant host of the present disclosure and collecting a culture containing the fusion protein.

The present disclosure provides a delivery system for delivering antigenic substances such as viruses, bacteria, fungi and tumors to CCR3-positive antigen-presenting cells. In the system, the antigen molecule is fused with chemokine CCL13, and a T2 tag is added at the end of the antigen molecule to further enhance the immunogenicity. The system may be in the form of a nucleic acid vector or fusion protein for the prevention or treatment of diseases caused by the antigen.

The present disclosure provides use of an antigen delivery system in the manufacture of a preventive or therapeutic vaccine, wherein the delivery system comprises a ligand CCL13 to bind to CCR3.

The present disclosure provides use of the fusion protein, nucleic acid, expression vector, host, fusion protein prepared by the method, and/or the culture containing the fusion protein prepared by the method of the present disclosure in the manufacture of a product for preventing and/or treating a disease.

The present disclosure further provides a product for preventing and/or treating a disease, comprising the fusion protein, nucleic acid, expression vector, host, fusion protein prepared by the method, and/or the culture containing the fusion protein prepared by the method.

The present disclosure also provides a method for preventing and/or treating a disease, comprising administering the product for preventing and/or treating a disease of the present disclosure.

In the present disclosure, the preventing and/or treating specifically includes increasing antibody level in serum, preventing tumor formation, inhibiting tumor growth, and improving immune response ability of the body against tumors.

In the present disclosure, the disease includes diseases caused by viruses and/or pathogenic microbes, or the disease is a tumor.

In the present disclosure, the product for preventing and/or treating a disease includes a medicine and/or a vaccine. In the present disclosure, the vaccine is selected from the group consisting of a DNA vaccine, a recombinant protein vaccine and an mRNA vaccine.

In the present disclosure, the administering includes oral administration, injection and/or electroporation.

Relevant studies and multiple experiments have shown that not all CC family chemokine members fused with antigen molecules can enhance immunogenicity. Only some are used to be fused with antigen molecules to enhance the immunogenicity of antigens, initiate a stronger immune response, and provide options for more potent vaccine preparations. For example, relevant studies have indicated that the fusion of 4-1BBL-S, 4-1BBL-Fc or CD80-Fc with antigen molecules can greatly enhance the immunogenicity of antigens, but the fusion of GM-CSF, mlL-23, or IL-15SAG1 with antigen molecules does not have the effect of enhancing immunogenicity.

In the present disclosure, the chemotactic binding ability of CCL13 to surface receptors on immune cells such as DCs is utilized to transport and cross-present various antigenic proteins to the surface of DCs, which improves the efficiency of phagocytosis, processing and presentation of various antigenic proteins by DCs, and improves the effect of preventing and/or treating related diseases. The T2 fragment of the present disclosure has been determined to have an extremely strong immune enhancement effect, and can further stimulate humoral and cellular immune responses in the process of promoting antigen presentation, finally achieving the effect of inhibiting the growth of related tumors.

### BRIEF DESCRIPTION OF DRAWINGS

In order to more clearly illustrate the specific embodiments of the present disclosure or the technical solutions in the prior art, the following will briefly introduce the drawings used in the description of the specific embodiments or the prior art. Apparently, the drawings in the following description are some embodiments of the present disclosure. For those of ordinary skill in the art, other drawings can also be obtained according to these drawings without making creative effort:

FIG. 1 shows the analysis of the ability of CCL13 to chemoattract various professional antigen-presenting cells.

FIG. 2 shows the vector maps of nucleotides encoding the fusion proteins; wherein A shows the plasmid map of pVR-CCL13-E6E7-T2, B shows the plasmid map of pVR-CCL13-E6E7, and C shows the plasmid map of pVR-E6E7.

FIG. 3 shows the expression of the target genes of the three plasmids pVR-CCL13-E6E7-T2, pVR-CCL13-E6E7 and pVR-E6E7, which is obtained by detecting the expression of the nucleotide encoding the fusion protein with a Flag tag at the C-terminal using Western blot.

FIG. 4 shows the timeline of the prophylactic immunization performed on mice with different fusion genes for later testing of specific T cell responses.

FIG. 5 shows the results of flow cytometric detection of specific T cell responses after immunization of mice with different fusion genes.

FIG. 6 shows the timeline of the tumor inoculation performed on mice with different fusion genes, mRNA and protein vaccines respectively for humoral immunity.

FIG. 7 shows the tumor measurement results of immunization in each group, wherein a shows the tumor measurement results of immunization in female mice; and b shows the tumor measurement results of immunization in male mice.

FIG. 8 shows the timeline of tumor inoculation in mice with different fusion genes, mRNA and protein vaccines for therapeutic immunization.

FIG. 9 shows the volume of cell tumors treated by each group.

### DETAILED DESCRIPTION

The present disclosure provides use of CCL13. Those skilled in the art can learn from the content herein and appropriately improve the process parameters for realization. It should be particularly pointed out that all similar replacements and modifications are apparent to those skilled in the art, and they are all considered to be included in the present disclosure. The method and use of the present disclosure have been described through preferred embodiments, and it is apparent that relevant persons can make changes or appropriate modifications and combinations of the methods and use herein without departing from the content, spirit and scope of the present disclosure to implement and apply the present disclosure.

Unless otherwise defined, all technical and scientific terms used herein have the same meaning as commonly understood by those of ordinary skill in the art. For definitions and terms in the art, those skilled can refer to Current Protocols in Molecular Biology (Ausubel). The standard three- and/or one-letter code used for expressing one of 20 common L-amino acids in the art is adopted as the abbreviation of an amino acid residue.

The CCL13 of the present disclosure is an important chemokine in the human body, belonging to the CC chemokine family, and can be specifically expressed at a high level in human macrophages and T cells. The present disclosure shows that CCL13 can be used to deliver substances to professional antigen-presenting cells, especially DCs, so as to enhance the presentation effect of DCs.

In the present disclosure, the CCL13 can be a humanized fragment, or fragments from other animals, such as murine, rabbit's, simian and porcine fragments. It can be a complete CCL13, or a fragment or mutant with CCL13 activity, which is not limited by the present application. In the examples of the present application, humanized CCL13 was used as the experimental object to demonstrate the improvement of the antigen presentation effect of CCL13. The humanized CCL13 has an amino acid sequence of

(SEQ ID NO: 3)
MKVSAVLLCLLLMTAAFNPQGLAQPDALNVPSTCCFTFSSKKISLQRLK
SYVITTSRCPQKAVIFRTKLGKEICADPKEKWVQNYMKHLGRKAHTLKT.

In the fusion protein of the present disclosure, the T2 fragment is modified from a short peptide at the C-terminal of bacteriophage T4 fibrin, and is an exogenous sequence in terms of species source. This sequence is completely absent in the human body, and will not cause problems of killing other human proteins after immune enhancement. It has been reported that this sequence can promote the trimerization of certain proteins under certain conditions. In the present disclosure, it is found that the T2 fragment, as an artificially modified polypeptide sequence, has an immune enhancing effect in the human body. In the present disclosure, the T2 fragment consists of 31 amino acids, and has a sequence of PGSGYIPEAPRDGQAYVRK DGEWVLLSTFLG (SEQ ID NO: 4).

The fusion protein of the present disclosure comprises at least one antigen. For example, it may comprise 1, 2, 3, 4, 5, 6, 7, 8, 9, 10 or more antigens. In the present disclosure, experiments were conducted on the fusion effect of one or two antigens, which all showed good results.

The antigen of the present disclosure is derived from viruses, pathogenic microbes and/or tumors. In the present disclosure, the antigen may be proteins derived from viruses, pathogenic microbe and/or tumors. In some embodiments, the antigen is derived from capsid proteins or non-structural proteins of viruses, membrane proteins or flagellin of pathogenic microbes, or surface antigens of tumors. It may be a complete fragment or an antigenic determinant thereof. It may contain only one antigenic determinant, or may be composed of multiple antigenic determinants in series, or may contain two or more repeats in series of one antigenic determinant.

In the present disclosure, the virus is selected from the group consisting of HPV, EBV, HCV, HIV, HBV, VZV, a coronavirus, and a combination thereof.

In the present disclosure, the tumor is selected from the group consisting of liver cancer, cervical cancer, ovarian cancer, lung cancer, head and neck cancer, prostate cancer, breast cancer, blood cancer, ovarian cancer, colorectal cancer, and a combination thereof.

In some embodiments, the antigen is E2, E5, E6 and/or E7 protein of HPV or a mutant epitope thereof. The HPV includes viruses of various HPV subtypes, for example, HPV6, HPV11, HPV16, HPV18, HPV31, HPV33, HPV35, HPV39, HPV45, HPV51, HPV52, HPV56 and/or HPV58.

In some embodiments, the antigen is LMP1, LMP2, EBNA1 of EB virus or a mutant epitope thereof.

In some embodiments, the antigen is S protein, N protein, E protein, M protein of a coronavirus or an epitope thereof. The coronavirus is SARS virus, MERS virus and/or COVID-19.

In some embodiments, the antigen is GPC3 protein and/or AFP protein of liver cancer.

In some embodiments, the antigen is PSA, PSMA, PSCA, PAP and/or STEAP1 of prostate cancer.

In some embodiments, the antigen is a dominant epitope of Her2/neu and/or BCAR3 of breast cancer.

In some embodiments, the antigen is MAGE-A3, ISR2, NY-ESO-1, Melan A, gp100, Tyrosinase, TRP1 and/or TRP2 of melanoma.

In some embodiments, the antigen is immunoglobulin idiotype, immunoglobulin K-chain and/or immunoglobulin λ-chain of blood cancer.

In some embodiments, the antigen is AIM2, HT001, TAF1B, Micoryx and/or TGFβRII of colorectal cancer.

In some embodiments, the antigen is folate receptor-a of ovarian cancer.

In some embodiments, the antigen is P53, IDH1/2, BAGE, GAGE1, GAGE2, CAG3, RAGE, CEA, CDK4, CASP-8, ras, bcr/abl and/or MUC-1 of various proto-oncogenes, anti-oncogenes and/or tumor-specific antigens.

In the present disclosure, the chemotactic binding ability of CCL13 to surface receptors on immune cells such as DCs is utilized to transport and cross-present the above antigenic proteins to the surface of DCs, which improves the efficiency of phagocytosis, processing and presentation of various antigenic proteins by DCs, and improves the effect of preventing and/or treating related diseases. In previous preliminary experiments, the presentation efficiency of multiple antigens of the above proteins including E6 or E7 protein of HPV16 has been demonstrated to be improved by CCL13. In the embodiments of the present disclosure, E6 and E7 proteins of HPV16 are taken as examples to prove that CCL13 can improve the efficiency of antigen protein presentation, and other proteins fused with CCL13 can also have a good effect.

In the present disclosure, in order to ensure smooth folding of each functional fragment in the fusion protein without being affected by steric hindrance, a linker is added between the fragments, wherein the linker between CCL13 and the antigenic protein of HPV is GGGGGSGGGGG. Different antigens can be linked by (GsS), and/or AGA.

In the present disclosure, in order to improve the expression effect of the fusion protein, a signal peptide that promotes the secretion of the fusion protein to the extracellular space is added to the N-terminal of CCL13. In some embodiments, the signal peptide is an IgE signal peptide. Specifically, it has an amino acid sequence of MDWTWILFLVAAATRVHS (SEQ ID NO: 5).

In the present disclosure, in order to facilitate the purification of the fusion protein, a tag is added to the C-terminal of the fusion protein. The tag is selected from recombinant protein purification tags well known in the art. In some embodiments, the tag is DYKDDDDK.

In some specific embodiments, the fusion protein has a structure which, from N-terminal to C-terminal, sequentially comprises an IgE signal peptide, a humanized CCL13 protein sequence, a linker sequence (GGGGGSGGGGG), an E6 E7 protein sequence, a T2 fragment sequence, and a Flag tag sequence. Specifically, it has an amino acid sequence set forth in SEQ ID NO: 15.

In the present disclosure, the nucleic acid encoding the protein may be DNA, RNA, cDNA or PNA. In an embodiment of the present disclosure, the nucleic acid is in the form of DNA or RNA. The DNA form includes cDNA, genomic DNA or synthetic DNA. The DNA may be single-stranded or double-stranded. The nucleic acid can comprise nucleotide sequences that have different functions, for example, a coding region and a non-coding region such as a regulatory sequence (e.g., a promoter or a transcription terminator). The nucleic acid can be linear or circular in topology. The nucleic acid can be, for example, a portion or a fragment of a vector, such as an expression or cloning vector. The nucleic acid may be obtained directly from natural sources, or may be prepared with the aid of recombinant, enzymatic or chemical techniques. The RNA form is mRNA obtained by gene transcription or the like.

In the present disclosure, the DNA sequence for expressing the fusion protein is optimized, and such optimization includes but is not limited to: codon usage bias, elimination of secondary structures (such as hairpin structures) that are not conducive to expression, changes in GC content, CpG dinucleotide content, secondary structure of mRNA, cryptic splice sites, early polyadenylation sites, internal ribosome entry and binding sites, negative CpG islands, RNA instability region, repeats (direct repeats, reverse repeats, etc.) and restriction sites that may affect cloning.

The prevention of the present disclosure refers to reducing the risk of tumor occurrence by administering the drug of the present disclosure before the tumor occurrence. The treatment of the present disclosure refers to inhibiting tumor growth, reducing tumor volume or delaying tumor growth by administering the drug of the present disclosure after tumor occurrence. In the examples of the present disclosure, the mouse transplanted tumor cell TC-1 is used as the experimental object to demonstrate the effect of the fusion protein vaccine.

The present disclosure further provides a transcription unit of the fusion protein, and the transcription unit refers to the DNA sequence beginning from the promoter and ending at the terminator. Regulatory fragments may also be comprised on either side of or between the promoter and terminator, and may include promoters, enhancers, transcription termination signals, polyadenylation sequences, origins of replication, nucleic acid restriction sites, and homologous recombination sites, such as promoter enhancers and poly(A) signals, which are operably linked to the nucleic acid sequence. The transcription unit provided by the present disclosure comprises a CMV or CMV/R promoter, a CMV enhancer and a nucleic acid fragment encoding the fusion protein.

The expression vector of the present disclosure refers to a recombinant nucleic acid vector, which is a recombinant DNA molecule that comprises a desired coding sequence and suitable nucleic acid sequences necessary for the expression of an operably linked coding gene in a specific host organism. Nucleic acid sequences necessary for expression in prokaryotic cells include a promoter with an optional operator sequence, a ribosome binding site and possibly other sequences. It is known that a promoter, enhancer, terminator and polyadenylation signals are used in prokaryotic cells. Once transformed into a suitable host, the vector can replicate and function independently of the host genome, or, in some cases, it can be integrated into the genome. In this specification, “plasmid” and “vector” are sometimes used interchangeably because plasmid is currently the most commonly used form of vector. However, the present disclosure is intended to include such other forms of expression vectors, which serve equivalent functions, and are or will become known in the art, including but not limited to: plasmids, phage particles, viral vectors and/or potential genomic insertions. In a specific embodiment, the nucleic acid encoding the fusion protein provided by the present disclosure can be constructed in various eukaryotic expression vectors. For example, the vector backbone can be a pVAX1 series vector, or a pVR series vector (see Chinese patent ZL 202110624820.8).

The host cell of the present disclosure is a prokaryotic or eukaryotic host containing a nucleic acid vector and/or a target gene. The host cells are transformed or transfected with vectors constructed using recombinant DNA techniques. Such transformed host cells are capable of replicating the vector encoding the protein or expressing the desired protein.

In the embodiments of the present disclosure, the fusion protein is prepared by a method of inducing the expression of the recombinant host, and the obtained culture can be bacteria, cells and culture solution obtained from culture, or substances obtained by extraction and/or purification from the above cultures.

The product for preventing and/or treating a disease provided by the present disclosure comprises the fusion protein, nucleic acid, expression vector, host, the fusion protein prepared by the method, and/or the culture containing the fusion protein prepared by the method. In the present disclosure, the product for preventing and/or treating a disease includes a medicine and/or a vaccine. The vaccine further comprises a pharmaceutically acceptable carrier, an excipient and/or an adjuvant. The medicine further comprises a pharmaceutically acceptable auxiliary material.

The prevention of the present disclosure refers to reducing the risk of disease occurrence by administering the product for preventing and/or treating a disease of the present disclosure before the disease occurrence. The treatment of the present disclosure refers to improving a disease, inhibiting the development of a disease, and restoring patients to health by administering the product for preventing and/or treating a disease of the present disclosure after the disease occurrence. For example, the use of the product of the present disclosure in preventing and/or treating tumors can increase the level of antibodies in serum, inhibit tumor growth, reduce tumor volume or delay tumor growth. In the examples of the present disclosure, the mouse transplanted tumor cell TC-1 is used as the experimental object to demonstrate the effect of the fusion protein vaccine, and good effects were obtained.

In the examples of the present disclosure, the amino acid sequences of the fragments involved and the coding nucleic acid fragments are shown in Table 1:

TABLE 1
Amino acid sequences and coding nucleic acid fragments involved in the present
disclosure

| | |
|---|---|
| SEQ ID NO: 1 | Amino acid sequence of E6 protein of HPV16 |
| SEQ ID NO: 2 | Amino acid sequence of E7 protein of HPV16 |
| SEQ ID NO: 3 | Amino acid sequence of CCL13 |
| SEQ ID NO: 4 | Amino acid sequence of T2 fragment |
| SEQ ID NO: 5 | IgE signal peptide |
| SEQ ID NO: 6 | Nucleotide sequence of plasmid pVR-CCL13-E6E7-T2 |
| SEQ ID NO: 7 | Nucleotide sequence of plasmid pVR-CCL13-E6E7 |
| SEQ ID NO: 8 | Nucleotide sequence of plasmid pVR-E6E7 |
| SEQ ID NO: 9 | DNA sequence encoding the fusion protein in plasmid pVR-CCL13- |
| | E6E7-T2 |
| SEQ ID NO: 10 | DNA sequence encoding the fusion protein in plasmid pVR-CCL13-E6E7 |
| SEQ ID NO: 11 | DNA sequence encoding the fusion protein in plasmid pVR-E6E7 |
| SEQ ID NO: 12 | mRNA sequence encoding the fusion protein in plasmid |
| | pVR-CCL13-E6E7-T2 |
| SEQ ID NO: 13 | mRNA sequence encoding the fusion protein in plasmid pVR-CCL13-E6E7 |
| SEQ ID NO: 14 | mRNA sequence encoding the fusion protein in plasmid pVR-E6E7 |
| SEQ ID NO: 15 | Amino acid sequence of the fusion protein in plasmid pVR-CCL13-E6E7- |
| | T2 |
| SEQ ID NO: 16 | Amino acid sequence of the fusion protein in plasmid pVR-CCL13-E6E7 |
| SEQ ID NO: 17 | Amino acid sequence of the fusion protein in plasmid pVR-E6E7 |

The reagents and consumables used in the present disclosure are all common commercially available products and can be purchased in the market. The present disclosure will be further illustrated below in conjunction with examples:

### Example 1

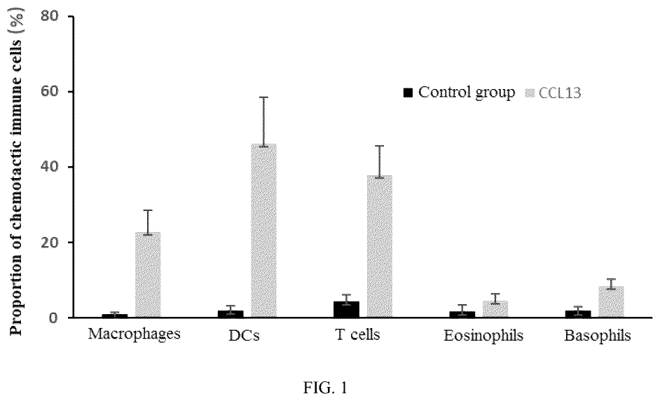

Monocytes, T cells, eosinophils, and basophils were isolated from mouse bone marrow and peripheral blood. Among them, the bone marrow monocytes were added with M-CSF, GM-CSF, and IL4 to induce differentiation into macrophages and DCs for chemotaxis experiments. The above isolated or induced-differentiated cells were placed in the upper chamber of a chemotaxis chamber (Transwell carbonate membrane chamber: 5 μm; Costar, Cat: 3422), and the cells were added at 1×10⁶ cells/100 μL/well based on the previous work in the laboratory. A spontaneous migration control group and a CCL13 cytokine group were set up simultaneously, and added with the same number of cells. There were 5 replicates in each group. The purified recombinant (E. coli) murine CCL13 protein was used as chemokine CCL13. According to the previous work in the laboratory, 200 ng/mL was the optimal dose for chemotactic efficiency. After 4 h, the cells in the lower chemotaxis chamber were collected, and the chemotactic ability of CCL13 to various immune cells was analyzed by flow cytometry. The results show that CCL13 could effectively recruit various immune cells from the upper chamber to the lower chamber (P<0.001) (FIG. 1).

### Example 2 Design of Fusion Gene Vaccine and Construction of Mammalian Expression Plasmid

Construction of plasmid pVR-E6E7: The E6 and E7 protein sequences of human papillomavirus subtype HPV16 were preceded by an IgE signal peptide with an amino acid sequence of MDWTWILFLVAAATRVHS, and followed by a Flag tag consisting of 8 amino acids of DYKDDDDK.

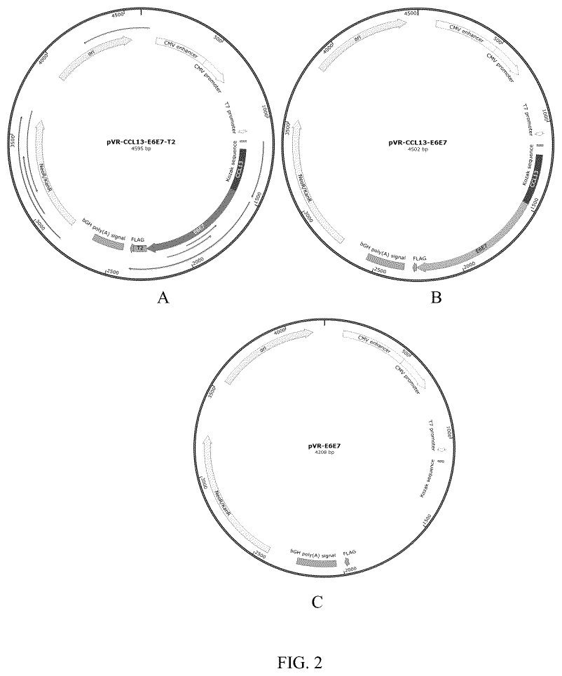

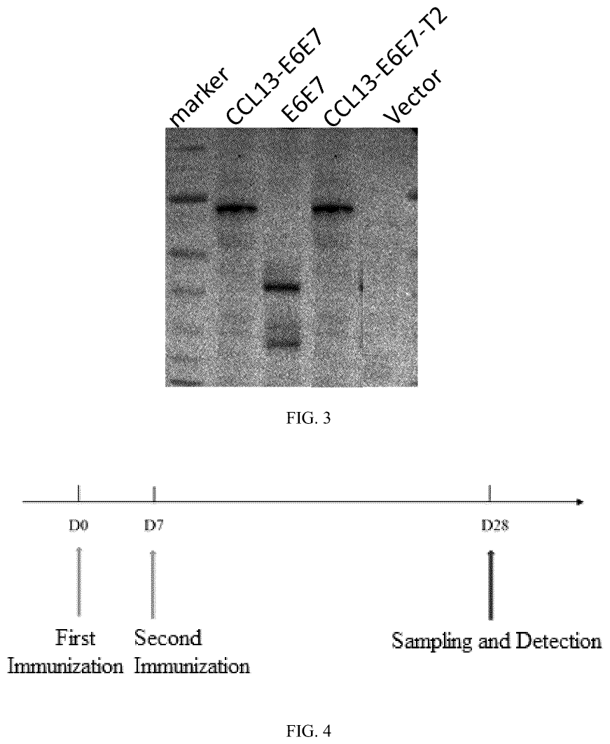

The finally obtained fusion protein, from N-terminal to C-terminal, sequentially comprised an IgE signal peptide, an E6 protein sequence, an E7 protein sequence, and a Flag tag sequence, as shown in A in FIG. 2.

The amino acid sequence of the fusion protein was subjected to codon optimization for expression preference of mammalian cells. The gene sequence of the fusion protein, determined as SEQ ID NO: 10, was then synthesized and inserted into the corresponding polyclonal sites region of pVR plasmid vector so as to express the fusion protein in the correct codon translation sequence. The finally constructed plasmid was named plasmid pVR-CCL13-E6E7, as shown in B in FIG. 2.

Construction of plasmid pVR-CCL13-E6E7: Similarly, the finally constructed fusion gene, from N-terminal to C-terminal, sequentially comprised an IgE signal peptide, a humanized CCL13 protein sequence, a linker sequence (GGGGGSGGGGG), an E6 protein sequence, an E7 protein sequence, and a Flag tag sequence.

The amino acid sequence of the fusion protein was subjected to codon optimization for expression preference of mammalian cells. The gene sequence of the fusion protein, determined as SEQ ID NO: 10, was then synthesized and inserted into the corresponding polyclonal sites region of pVR plasmid vector so as to express the fusion protein in the correct codon translation sequence. The finally constructed plasmid was named plasmid pVR-CCL13-E6E7, as shown in B in FIG. 2.

Construction of plasmid pVR-CCL13-E6E7-T2: the final fusion gene, from N-terminal to C-terminal, sequentially comprised an IgE signal peptide, a humanized CCL13 protein sequence, a linker sequence (GGGGGSGGGGG), an E6 protein sequence, an E7 protein sequence, a T2 fragment sequence, and a Flag tag sequence. The fusion protein CCL13-E6E7-T2 was first constructed, then connected with the IgE signal peptide with an amino acid sequence of MDWTWILFLVAAATRVHS at the N-terminal, and connected with the Flag tag sequence with an amino acid sequence of DYKDDDDK at the C-terminal.

The amino acid sequence of the fusion protein was subjected to codon optimization for expression preference of mammalian cells. The gene sequence of the fusion protein, determined as SEQ ID NO: 8, was then synthesized and inserted into the corresponding polyclonal sites region of pVR plasmid vector so as to express the fusion protein in the correct codon translation sequence. The finally constructed plasmid was named plasmid pVR-CCL13-E6E7-T2, as shown in C in FIG. 2.

Specifically, the plasmid patterns constructed in the experiments in this example are actually: pVR-CCL13-antigen-T2, pVR-CCL13-antigen and a control plasmid pVR-antigen. In the experiment of this example, the E6E7 fusion protein of HPV16 subtype was used as the antigen.

### Example 3 Western Blot Detection of Intracellular Expression of Fusion Gene or Protein Vaccine

HEK293T cells were transfected with the recombinant plasmid pVR-E6E7, pVR-CCL13-E6E7, and pVR-CCL13-E6E7-T2 by PEI transfection method, and the transfection method for vectors containing other antigens was the same.

The cells were subcultured 24 h before transfection and controlled at a density of 60%-80% during transfection. Opti-MEM medium and plasmids were added to a centrifuge tube at a ratio as shown in Table 2:

TABLE 2
Plasmid transfection

| Culture plate type | Opti-MEM Medium | Plasmid DNA |
|---|---|---|
| 6-well plate | 200 μL | 3 μg |
| 10 cm dish | 1 mL | 8 μg |

The prepared PEI transfection reagent was added to the diluted DNA at a ratio of PEI (μg): total DNA (μg) of 3:1. The resulting mixture was mixed well for 10 s-15 s, and left to stand at room temperature for 15 min. Cells were slowly dropwise added with the above solution with a pipette, and incubated in an incubator for 48 h. Cells were slowly dropwise added with the above solution with a pipette, and incubated in an incubator for 48 h. Then the well-mixed NP40 lysate containing protease inhibitors was added. Cells were scraped off with a cell scraper, transferred to a 1.5 mL centrifuge tube, and placed on a shaker at 4° C. for 20 min of lysis. The sample was centrifuged at 12000 rpm for 20 min at 4° C., and the supernatant was transferred to a new centrifuge tube. 50 μg of the quantified protein sample was added into a centrifuge tube, added with 4× protein loading buffer, mixed well, centrifuged, and boiled at 95° C. for 5 min. The samples were detected by Western Blot, and the results show that the plasmids can be expressed normally in mammalian cells.

### Example 4

The effects of the CCL13 chemokine and T2 fragment on cell-specific T cell responses induced by the fusion gene vaccine were explored. The vaccines comprising antigens of HPV16 E6 and E7 proteins were taken as an example and the steps for inducing T cell responses by vaccines comprising other antigens were the same.

Given that the fusion gene can be normally expressed in mammalian cells, plasmids pVR-CCL13-E6E7-T2, pVR-CCL13-E6E7, and pVR-E6E7 were extracted separately, and the mice were immunized with the plasmids of 25 μg by an in vivo gene transfer instrument from TERESA. There were four groups including a negative control PBS group, 5 mice in each group. The mice were immunized according to the immunization strategy marked in the timeline in FIG. 4, and blood was collected from the mice in each group on D14 for test.

100 μL of fresh blood from the mice was taken, added with an appropriate amount of anticoagulant (sodium heparin), and centrifuged at 3000 rpm for 5 min on a high-speed freezing centrifuge. The supernatant was gently discarded, and the remaining pellet was shaken to disperse, added with 1 mL of red blood cell lysis buffer for 1 min of lysis, and centrifuged at 3000 rpm for 5 min. The supernatant was discarded, and each sample was added with 1 mL of PBS, mixed with a vortex shaker, and centrifuged at 1500 rpm for 5 min in a high-speed freezing centrifuge. The supernatant was discarded, and the white cell mass at the bottom was reserved. Anti-CD8 antibody and HPV16 E7 antibody were diluted with 1640 medium, where every 100 μL of 1640 medium was added with 0.2 μL of Anti-CD8 antibody or 1 μL of HPV16 E7 (E7-tetramer) antibody. 100 μL of staining solution was added to each sample. The cell mass was dispersed with a 200 μL pipette for fully staining of the cells, and the staining was conducted at 4° C. for 1 to 2 h. After staining, each sample was added with 1 mL of PBS, and centrifuged at 3000 rpm for 5 min in a high-speed freezing centrifuge. The supernatant was discarded, and the white cell mass at the bottom was reserved. Each sample was added with 200 μL of PBS, and the cell mass was dispersed with a 200 μL pipette to make a cell suspension, which was then transferred to a flow tube. Flow staining: cd8-pe; E7-tetramer-Fitc. The results are shown in the figure and indicate that in the comparison of the same dose groups, the number of E7-specific T cells in the E6E7 group, CCL13-E6E7 group, and CCL13-E6E7-T2 group was much higher than that in the control group. In the comparison of the same dose groups, the number of E7-specific T cells in the CCL13-E6E7-T2 group was significantly higher than that in the E6E7 group and the CCL13-E6E7 group. In the comparison of the same dose groups, the number of E7-specific T cells in the CCL13-E6E7 group was significantly higher than that in the E6E7 group. This indicates that chemokine CCL13 can indeed effectively induce the binding of E6E7 antigen and specific immune cells, thereby promoting the formation of T cells. In addition, the addition of T2 fragment at the C-terminal of the antigen can significantly enhance the effect of CCL13 presenting antigen molecules.

### Example 5

The effects of fusion gene DNA, mRNA and protein vaccines on inducing humoral immune responses were explored. The protein vaccines comprising antigens of HPV16 E6 and E7 proteins were taken as an example, and the steps for the demonstration of tumor intervention effect of vaccines comprising other antigens were the same.

Given that the fusion gene can be normally expressed in mammalian cells, plasmids pVR-CCL13-E6E7-T2 and pVR-E6E7 were extracted separately, and the mice were immunized with the plasmids by an in vivo gene transfer instrument from TERESA. The mRNA vaccine of pVR-CCL13-E6E7-T2 was prepared by in vitro transcription, which was then encapsulated with an in vivo transfection reagent, in vivo-jet PEI, into lipid nanoparticles as a mRNA form vaccine (recorded as CCL13-E6E7-T2-mRNA).

The fusion protein was also purified as a protein vaccine, wherein the fusion protein corresponding to the plasmid pVR-CCL13-E6E7-T2 was recorded as CCL13-E6E7-T2.

After allografting of TC-1 cells, the inhibitory effect of the fusion gene vaccine on the growth of TC-1 transplanted tumor cells was observed.

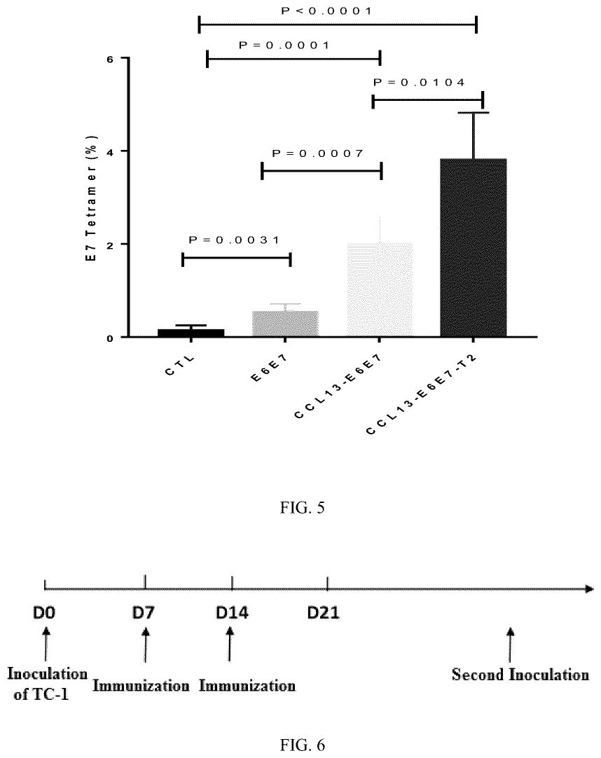

First, the tumor-forming conditions of TC-1 cells were explored to select the optimal dose. The mice were then subjected to plasmid electroporation, intramuscular injection of mRNA or subcutaneous injection of protein according to the immunization strategy marked in the timeline of FIG. 6. The preparation process of RNA vaccine is as follows:

5′UTR-CCL13-E6E7-3′UTR-A (120) was connected into the cloning vector pGEM-3Zf (+) (promega) to complete the construction of an in vitro transcription expression system, which was named pGEM-CCL13-E6E7. 5′UTR-CCL13-E6E7-T2-3′UTR-A (120) was connected into the cloning vector pGEM-3Zf (+) (promega) to complete the construction of an in vitro transcription expression system, which was named pGEM-CCL13-E6E7-T2. The in vitro transcription system had a UTR sequence as follows:

5-UTR (β-globin-2)
agagcggccgctttttcagcaagattaagcccagggcagagccatctat
tgcttacatttgcttctgacacaactgtgttcactagcaacctcaaaca
gacacc
3-UTR (2β-globin)
agctcgctttcttgctgtccaatttctattaaaggttcctttgttccct
aagtccaactactaaactgggggatattatgaagggccttgagcatctg
gattctgcctaataaaaaacatttattttcattgcagctcgctttcttg
ctgtccaatttctattaaaggttcctttgttccctaagtccaactacta
aactgggggatattatgaagggccttgagcatctggattctgcctaata
aaaaacatttattttcattgc

The recombinant plasmid was linearized. The single enzyme digestion reaction system is shown in the table below, and the reaction was conducted at 37° C. for 3 h.

| Component | Volume (μL) |
|---|---|
| Xho I restriction endonuclease | 4 |
| Recombinant plasmid | 12 μg |
| 10× endonuclease buffer | 5 |
| Water | Balance |
| Total | 50 |

### In Vitro Transcription Reaction:

T7-Flash Scribe™ Transcription Kit (Cell script) was use for in vitro transcription, and UTP was replaced with N1-Methylpseudouridine-5′-Triphosphate (Trilink Biotech) during the preparation of in vitro transcription system.

The reaction system is shown in the table below. After the first step of reaction was completed, the reaction system was kept at 35° C. for 30 min, and after the second step of reaction was completed, the reaction system was kept at 35° C. for 15 min.

mRNA Capping:

The kit ScriptCap™ Cap 1 Capping System (Cell script) was used. The reaction system is shown in the table below.

TABLE 4

| | |
|---|---|
| Component | Volume (μL) |

Step 1

| RNase free water | X |
|---|---|
| In vitro transcribed RNA, 50 μg-60 μg | ≤67 |
| Total volume | 67 |
| 15 min of reaction at 65° C. | |
| Step 2 | |
| 10× ScriptCap Caping buffer | 10 |
| 10 mM GTP | 10 |
| 20 mM SAM | 2.5 |
| ScripGuard RNase inhibitor | 2.5 |
| ScripCap 2-O-Methyltransferase (100 U/μL) | 4 |
| Total volume | 29 |

Step 3

| | |
|---|---|
| Reaction solution from Step 2 | 29 |
| ScriptCap Capping Enzyme (10 U/μL) | 4 |
| Heat-denatured RNA (Step 1) | 67 |
| Total volume | 100 |

30 min of reaction at 37° C.

Cap-mRNA purification: Purification was performed using the kit MEGAclear™ Kit Purification (Invitrogen).

### Preparation of Liposome Nanoparticles LNP-Man

DOTAP ((2,3-dioleoyl-propyl)trimethylammonium chloride), DOPE (dioleoylphosphatidylethanolamine), and DSPE-PEG2000 (distearoylphosphatidylethanolamine-polyethylene glycol 2000) were all purchased from Shanghai Advanced Vehicle Technology Co., Ltd. DSPE-PEG2000-Man was purchased from Xi'an Haoran Biotechnology Co., Ltd.;

Nanoparticles were prepared by rotary evaporation method. The specific operation process of preparation of LNP-Man is as follows:

1) DOTAP, DOPE, and DSPE-PEG2000-Man were sequentially added to a round bottom flask according to the molar ratio of 50:50:1. 6 mL of chloroform was added until the solid was fully dissolved.

2) Ultrasonication in a water bath was performed for 15 min.

3) The round-bottom flask was put into a rotary evaporator, and the dissolved matter in the round-bottom flask should be submerged below the water surface. Then rotary evaporation was performed at a speed of 100 rpm for 15 min.

4) The round bottom flask was detached, placed in a fume hood, and added with 8 mL of HEPES buffer to dissolve the film on the inner wall of the bottle.

5) Ultrasonication in a water bath was performed for 30 min.

6) The ultrasonicated solution was filtered with a 0.22 μm filter membrane three times to obtain the desired liposome nanoparticles LNP-Man.

### Preparation of LNPs/mRNA

The prepared cationic lipid nanomaterial LNP-Man and mRNA were mixed according to the set N/P=10:1 (molar ratio), and the required volume of LNPs and mRNA was calculated. LNPs and mRNA were respectively added with equal volumes of 10 mM HEPES buffer solution before mixing. The mixed LNPs/mRNA was shaken on a vortex shaker for 1 min, and left to stand at room temperature for 30 min.

Female and male C57BL6 mice aged 6-8 weeks (purchased from Vital River) were randomly divided into groups as follows:

TABLE 5
Animal grouping

| Group | Animal(s)/gender | Animal No./gender | |
|---|---|---|---|
| PBS | 5/♂ 5/♀ | 1-5/♂ | 6-10/♀ |
| pVR-E6E7 | 5/♂ 5/♀ | 11-15/♂ | 16-20/♀ |
| pVR-CCL13-E6E7-T2 | 5/♂ 5/♀ | 21-25/♂ | 26-30/♀ |
| CCL13-E6E7-T2-mRNA | 5/♂ 5/♀ | 31-35/♂ | 36-40/♀ |
| CCL13-E6E7-T2-protein | 5/♂ 5/♀ | 41-45/♂ | 46-50/♀ |

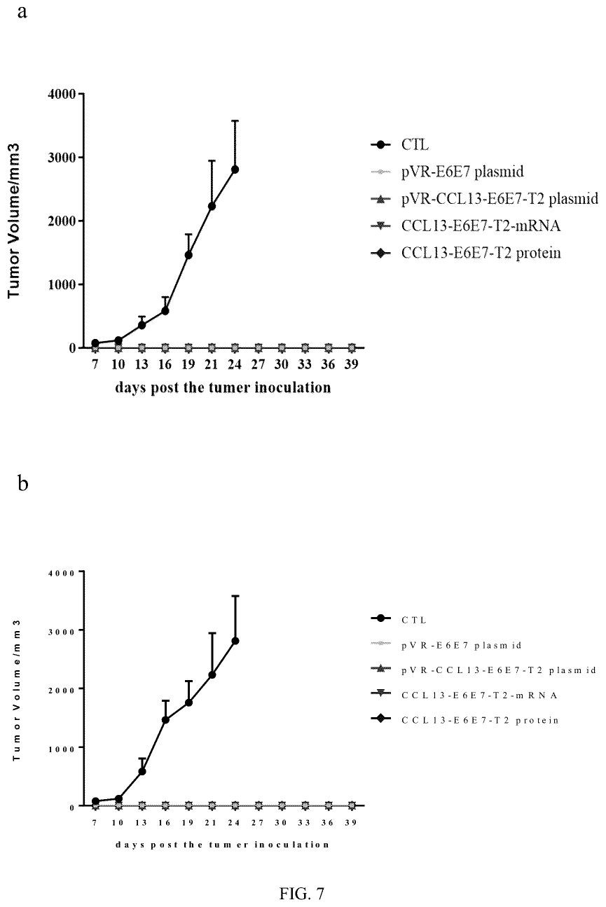

The right side near the inguinal lymph node of the mouse was depilated. The mice were directly injected with mRNA or protein, or intramuscularly injected with DNA vaccine supplemented with electrical pulse stimulation. Each mouse was injected at a dose of 25 μg, and at a frequency of once every two weeks, for a total of two injections. One week after the last injection, mice were inoculated with TC-1 tumor cells whose tumor-forming conditions had been explored. The tumor formation time was observed. The long diameter a and short diameter b of the tumor were measured every two days to calculate the tumor volume according to a×b×b/2, and the tumor growth curve was plotted. The results are shown in FIG. 7, which indicate that the injection of pVR-E6E7, pVR-CCL13-E6E7-T2, CCL13-E6E7-T2-mRNA and CCL13-E6E7-T2-protein vaccine can completely prevent tumor growth and achieve the effect of preventing tumor growth. In addition, mice in all experimental groups were subjected to tumor transplantation for the second time on D30, and the transplanted tumors still failed to form tumors. It shows that the four vaccines all have a good effect on preventing tumor growth.

### Example 6

The effects of fusion gene DNA, RNA and protein vaccines in treating tumors were explored. The protein vaccines comprising antigens of HPV16 E6 and E7 proteins were taken as an example, and the steps for the demonstration of tumor intervention effect of vaccines comprising other antigens were the same.

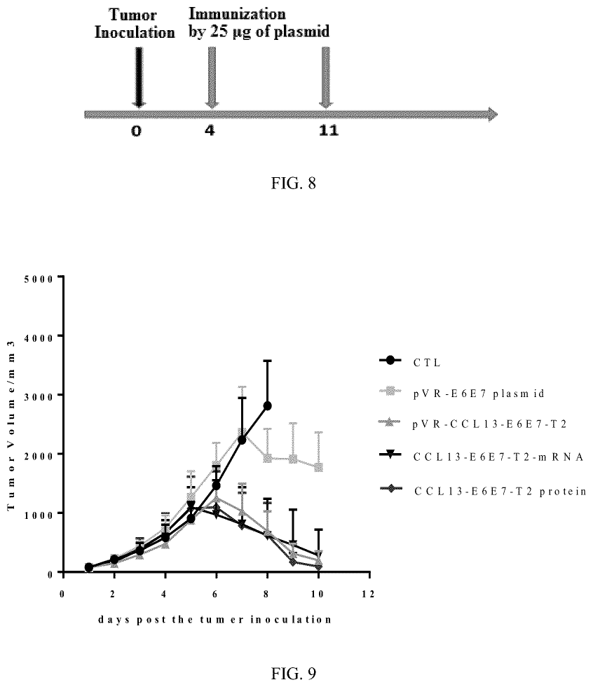

In view of the remarkable preventive effect of the four vaccines, the therapeutic effects of the vaccines were demonstrated. Mice were inoculated with TC-1 cells on DO, divided into groups on D4, and injected with vaccines on D4 and D11 at the same dose as that of the prevention group. The protocol is shown in FIG. 8. After that, the tumor size was measured twice or three times a week according to the same calculation method as above. The tumor growth curve is shown in FIG. 9. The results show that the four vaccines could all inhibit the growth of tumor cells to varying degrees, and the plasmid pVR-CCL13-E6E7-T2, CCL13-E6E7-T2-mRNA and CCL13-E6E7-T2 protein groups had a better therapeutic effect on tumors and could achieve the effect of completely eliminating tumors.

Finally, it should be stated that: the above embodiments are only intended for illustrating the technical solutions of the present disclosure rather than limiting the present disclosure. Although the present disclosure is illustrated in detail with reference to the embodiments described above, it should be understood by those skilled in the art that, modifications can still be made to the technical solutions recited in the embodiments described above, or equivalent substitutions can be made onto a part or all of the technical features of the technical solution. While such modifications or substitutions will not cause the essence of corresponding technical solutions to depart from the scope of the technical solutions of the embodiments of the present disclosure.

1. A method for improving antigen presentation effect, comprising administering to a subject in need thereof at least one of I)-VI);
I) a T2 fragment with an amino acid sequence set forth in SEQ ID NO: 4;
II) chemokine CCL13;
III) a fragment being more than 80% homologous with and functionally identical or similar to I) or II);
IV) a nucleic acid molecule encoding I) or II);
V) a nucleic acid molecule that is derived from the nucleotide sequence of the nucleic acid molecule in IV) by deletion, addition or substitution of one or more nucleotides and encode a protein functionally identical or similar to the nucleic acid molecule in IV);
VI) a nucleic acid molecule fully or partially complementary to V).

2. A fusion protein, comprising CCL13 and an antigen or comprising CCL13, an antigen and a T2 fragment.

3. The fusion protein according to claim 2, from N-terminal to C-terminal, sequentially comprising an IgE signal peptide, CCL13, a linker, the antigen and the T2 fragment.

4. The fusion protein according to claim 2, wherein the antigen is derived from a virus, pathogenic microbe and/or tumor;
the virus is selected from the group consisting of HPV, EBV, HCV, HIV, HBV, VZV, a coronavirus, and a combination thereof; and
the tumor is selected from the group consisting of liver cancer, cervical cancer, ovarian cancer, lung cancer, head and neck cancer, prostate cancer, breast cancer, blood cancer, ovarian cancer, colorectal cancer, and a combination thereof.

5. A nucleic acid encoding the fusion protein according to claim 2.

6. A nucleic acid fragment comprising the nucleic acid according to claim 5, 5′-UTR, 3′-UTR and 3′-end polyA.

7. An expression vector comprising a vector backbone and the nucleic acid according to claim 5.

8. A host transformed or transfected with the expression vector according to claim 7.

9. A method for producing the fusion protein according to claim 2, comprising culturing a host transformed or transfected with an expression vector comprising a vector backbone and a nucleic acid encoding the fusion protein and collecting a culture containing the fusion protein.

10. (canceled)

11. A product for preventing and/or treating a disease, comprising the fusion protein according to claim 2.

12. A method for preventing and/or treating a disease, comprising administering to a subject in need thereof the product according to claim 11.
